(19) United States
(12) Patent Application Publication
(10) Pub. No.: US 2014/0170088 A1
(43) Pub. Date: Jun. 19, 2014

(54) BARIUM ZIRCONIUM OXIDE (BaZrO3.BaCO3) NANOPARTICLES

(71) Applicants: SECRETARY, DEPARTMENT OF ELECTRONICS AND INFORMATION TECHNOLOGY (DeitY), New Delhi (IN); CENTRE FOR MATERIALS FOR ELECTRONICS TECHNOLOGY (C-MET), Pune (IN)

(72) Inventors: Bharat Bhanudas Kale, Pune (IN); Milind Vyankatesh Kulkarni, Pune (IN); Rajendra Popat Panmand, Pune (IN); Ujjwala Vilas Kawade, Pune (IN); Sanjay Krushnaji Apte, Pune (IN); Sonali Deepak Naik, Pune (IN); Jalindar Dnyandev Ambekar, Pune (IN); Ravindra Shravan Sonawane, Pune (IN); Ramadoss Marimuthu, Pune (IN); Dinesh Pundalik Amalnerkar, Pune (IN); Nilofer Shroff, New Delhi (IN); Sandip Chatterjee, New Delhi (IN)

(73) Assignees: SECRETARY, DEPARTMENT OF ELECTRONICS AND INFORMATION TECHNOLOGY (DeitY), New Delhi (IN); CENTRE FOR MATERIALS FOR ELECTRONICS TECHNOLOGY (C-MET), Pune (IN)

(21) Appl. No.: 14/107,161

(22) Filed: Dec. 16, 2013

(30) Foreign Application Priority Data
Dec. 18, 2012 (IN) 3550/MUM/2012

(51) Int. Cl. G21F 1/06; A61Q 17/04; G21F 3/00; A61K 8/28

(52) U.S. Cl. CPC G21F 1/06; A61K 8/28; A61Q 17/04; G21F 3/00
USPC 424/59; 252/478; 427/160; 427/2.1; 427/2.31; 428/402; 428/330; 442/133

(57) ABSTRACT

The present disclosure provides nanoparticles of barium zirconium oxide (BaZrO₃.BaCO₃) and a process for preparation thereof. The present disclosure further provides a coating formulation that chiefly comprises the BaZrO₃.BaCO₃ nanoparticles along with its method of preparation. Still further, the present disclosure provides X-ray retardant articles and an X-ray retardant preparation for topical application.

### CROSS REFERENCE TO RELATED APPLICATIONS

This application is filed under 35 USC 111 and hereby claims priority under 35 U.S.C. 119 from Indian patent application Serial no. 3550/MUM/2012 filed on Dec. 18, 2012 the disclosure of which is hereby incorporated herein by reference in its entirety.

### FIELD OF THE DISCLOSURE

The present disclosure relates to barium zirconium oxide (BaZrO₃.BaCO₃) nanoparticles.

### BACKGROUND

Nanoparticles are broadly defined as particles with at least one dimension ranging from 1 to 100 nanometres. As a virtue of their small size, nanoparticles are associated with plentiful advantages such as high stability, greater penetrability, greater solubility, high dielectric permittivity and therefore enhanced functionality. As a consequence, nanoparticles have a very wide spectrum of applications such as in pharmaceuticals, diagnostics, therapeutics, electronics, biomaterials and energy production that make this particular physical entity extremely attractive.

Nanostructured barium zirconium oxide units have been prepared from various methods such as solid state reactions, soft chemistry routes (polymer precursor), sol-gel processes and spray pyrolysis. Of these, solid state reactions are normally performed at extremely high temperatures such as 1300° C. and therefore cannot be used for thermolabile materials. Other processes such as high-energy dry grinding, laser ablation and spray pyrolysis usually require long synthesis times and employ sophisticated and expensive equipments.

Barium zirconium oxide nanostructures, once synthesized, may find varied applications such as use in optical devices, X-ray attenuation devices, vacuum tubes, pyrotechnics, pigments, paints and glass making. As for the use in the X-ray attenuation or shielding devices, conventionally, pure lead metal or lead oxide have been the most commonly used agents. However, lead being a toxic substance that chiefly targets the nervous system in adults as well as children it has to be used with great discretion. It can damage the nervous connections and cause blood and brain disorders. The greatest risk from lead poisoning is to children under the age of seven, whose developing bodies and brains are sensitive to even small amounts of lead. A directive on the restriction of the use of certain hazardous substances (RoHS), adopted by the European Union in 2003, is in line with this consequence. Of the six hazardous substances whose use is to be restricted, lead is the first one. Along with Europe, many other countries observe similar restrictions regarding the use of lead for various applications such as electrical and electronic equipment.

Bismuth (Bi), a free element that is 86% as dense as lead, has also been considered as a substitute for lead due to the low toxicity. However, prolonged exposure to Bi may lead to toxicity and therefore its use also needs to be strictly monitored. There is therefore a need to develop a less toxic, yet equally effective X-ray shielding material that can be used for multiple purposes.

The present disclosure envisages barium zirconium oxide nanoparticles and a method for preparation of the same; the method being comparatively fast and cost-effective. The present disclosure further envisages a coating formulation comprising barium zirconium oxide nanoparticles and a process for preparation of the same, where the formulation can be employed for varied applications including use in X-ray shielding devices, formulations and cosmeceuticals.

Definitions

As used in the present disclosure, the following words and phrases are generally intended to have the meaning as set forth below, except to the extent that the context in which they are used to indicate otherwise.

The expression ‘article’ means any cloth or garment or object or surface that includes aprons, gowns, scrubs, uniforms, gloves, caps, masks, curtains, sheets, fabrics, shoe covers, drapes, surgical pads, protective screens, thyroid collars, thyroid shields, desks, drawers, rooms, walls, partitions, panels, tables, chairs, cabinets and ceiling panels that can be used for multifarious applications.

Objects

Some of the objects of the present disclosure are discussed herein below:

It is an object of the present disclosure to provide nanoparticles of barium zirconium oxide (BaZrO₃.BaCO₃).

It is another object of the present disclosure to provide a process for the preparation of BaZrO₃.BaCO₃ nanoparticles.

It is yet another object of the present disclosure to provide a process for the preparation of BaZrO₃.BaCO₃ nanoparticles which is cost-efficient, rapid, high yielding and environment friendly.

It is still another object of the present disclosure to provide a coating formulation.

It is yet another object of the present disclosure to provide a coating formulation which is less toxic, chemically stable, water repellant, environmentally friendly and possessing X-ray attenuation properties.

It is still another object of the present disclosure to provide a process for the preparation of the coating formulation.

It is yet another object of the present disclosure to provide a process for the preparation of the coating formulation, which is simple and economical.

It is still another object of the present disclosure to provide an X-ray retardant article and a process for preparation of the same.

It is yet another object of the present disclosure to provide an X-ray retardant preparation for cosmetic or medicinal applications.

It is still another object of the present disclosure to ameliorate one or more problems of the prior art or to at least provide a useful alternative.

Other objects and advantages of the present disclosure will be more apparent from the following description when read in conjunction with the accompanying figures which are not intended to limit the scope of the present disclosure.

### SUMMARY

The present disclosure provides nanoparticles of barium zirconium oxide (BaZrO₃.BaCO₃); said nanoparticles characterized by particle size ranging between 50 and 300 nm, preferably between 100 and 250 nm and the X-ray diffraction pattern having 20 values at 21.17, 30.06, 37.19, 43.16, 53.46, 62.62, 71.14 and 79.15.

The present disclosure further provides a process for synthesizing barium zirconium oxide (BaZrO₃.BaCO₃) nanoparticles; said process comprising the following steps:
i. admixing at least one source of barium, at least one source of zirconium, at least one combustion agent, at least one thermal decomposition agent, at least one precipitation agent and at least one reducing agent under inert atmosphere to obtain a slurry; and
ii. subjecting said slurry to combustion at a temperature ranging between 500° C. and 800° C., preferably between 645° C. and 655° C., for a time period ranging between 1.0 and 5.0 hours, preferably between 2.5 and 3.5 hours to obtain BaZrO₃.BaCO₃ nanoparticles.

Typically, the source of barium is at least one selected from the group consisting of barium nitrate, barium chloride and barium acetate, preferably barium nitrate.

Typically, the source of zirconium is at least one selected from the group consisting of zirconium oxychloride, zirconium chloride and zircolyl nitrate, preferably zirconium oxychloride.

Typically, the mole ratio of the source of barium to the source of zirconium is 2:1.

Typically, the combustion agent is at least one selected from the group consisting of ammonium nitrate, hydroxylammonium nitrate and hexanitrodiphenylamine, preferably ammonium nitrate.

Typically, the thermal decomposition agent is at least one selected from the group consisting of glycine and N-methylglycine, preferably glycine.

Typically, the precipitation agent is at least one selected from the group consisting of urea, 1, 3-dimethylurea and isopropylideneurea, preferably urea.

Typically, the reducing agent is at least one selected from the group consisting of starch, acrylamide and carboxymethylcellulose, preferably starch, in an amount ranging between 10 and 30 mole %, preferably between 15 and 25 mole % with respect to the total moles of the source of barium.

Typically, the proportion of the combustion agent, the thermal decomposition agent and the precipitation agent is 1: 1: 5.

Typically, the inert atmosphere is carbon dioxide (CO₂) atmosphere.

Typically, the method-step of combustion involves addition of said slurry into a hot silica tube in a drop-wise fashion.

Typically, the BaZrO₃.BaCO₃ nanoparticles have particle size ranging between 50 and 300 nm, preferably between 100 and 250 nm.

The present disclosure even further provides a coating formulation comprising barium zirconium oxide (BaZrO₃.BaCO₃) nanoparticles having particle size ranging between 50 and 300 nm, preferably between 100 and 250 nm, in an amount ranging between 10% and 50%, preferably between 20% and 30% of the total mass of a vehicle, at least one color guard and at least one vehicle.

Typically, the color guard is at least one selected from the group consisting of light aliphatic solvent naphtha (64742-89-8), n-hexane (110-54-3), xylene (1330-207), acetone, ethyl benzene (100-41-4), silica amorphous, fumed, crystalline free (1129455-52-5) carbon black (133-86-4) [Loctite make], epoxy, nitrocellulose and ethyl cellulose in an amount ranging between 5% and 25%, preferably between 8% and 12% of the total mass of the vehicle.

Typically, the vehicle is at least one selected from the group comprising ethyl methyl ketone, amyl acetate and acetone, preferably ethyl methyl ketone.

The present disclosure still further provides a process for the preparation of a coating formulation comprising barium zirconium oxide (BaZrO₃.BaCO₃) nanoparticles having particle size ranging between 50 and 300 nm, preferably between 100 and 250 nm, at least one color guard and at least one vehicle; said process comprising the following steps:
i. milling said BaZrO₃.BaCO₃ nanoparticles in an amount ranging between 10% and 50%, preferably between 20% and 30% of the total mass of the vehicle to obtain a mass; and
ii. admixing said mass and the color guard in an amount ranging between 5% and 25%, preferably between 8% and 12% of the total mass of the vehicle, in the vehicle, followed by milling to obtain said coating formulation.

Typically, milling is carried out for a time period ranging between 12 and 48 hours, preferably between 21 and 26 hours.

The present disclosure even further provides a process for the preparation of an X-ray retardant article; said process comprising the following steps:
i. contacting said article with a coating formulation comprising barium zirconium oxide (BaZrO₃.BaCO₃) nanoparticles having particle size ranging between 50 and 300 nm, preferably between 100 and 250 nm, in an amount ranging between 10% and 50%, preferably between 20% and 30% of the total mass of a vehicle, at least one color guard and at least one vehicle, to obtain a coated article; and
ii. annealing the coated article by hot air at a temperature ranging between 20° C. and 80° C., preferably between 50° C. and 60° C. for a time period ranging between 0.5 and 5 minutes, preferably between 0.5 and 2 minutes obtain an X-ray retardant article.

Typically, the article is at least one selected from the group comprising aprons, gowns, scrubs, uniforms, gloves, caps, masks, curtains, sheets, fabrics, shoe covers, drapes, surgical pads, protective screens, thyroid collars, thyroid shields, desks, drawers, rooms, walls, partitions, panels, tables, chairs, cabinets and ceiling panels.

Typically, the article is contacted with the coating formulation by at least one method selected from the group comprising applying, spraying, dipping, incorporating, brushing and painting.

Typically, the color guard is at least one selected from the group comprising light aliphatic solvent naphtha (64742-89-8), n-hexane (110-54-3), xylene (1330-207), acetone, ethyl benzene (100-41-4), silica amorphous, fumed, crystalline free (1129455-52-5) carbon black (133-86-4) [Loctite make], epoxy, nitrocellulose and ethyl cellulose, in an amount ranging between 5% and 25%, preferably between 8% and 12% of the total mass of the vehicle.

]Typically, the vehicle is at least one selected from the group comprising ethyl methyl ketone, amyl acetate and acetone, preferably ethyl methyl ketone.

The present disclosure still further provides an X-ray retardant article, prepared by the process as claimed in claim 19, wherein said article is at least one article selected from the group comprising aprons, gowns, scrubs, uniforms, gloves, caps, masks, curtains, sheets, fabrics, shoe covers, drapes, surgical pads, protective screens, thyroid collars, thyroid shields, desks, drawers, rooms, walls, partitions, panels, tables, chairs, cabinets and ceiling panels.

Typically, said article is made up of at least one material selected from the group consisting of cotton, epoxy resin, acrylic, lyocell, nylon, polyester, rubber, wood, glass, metal, metal alloys and paper.

The present disclosure even further provides an X-ray retardant preparation comprising:
i. barium zirconium oxide (BaZrO₃.BaCO₃) nanoparticles having particle size ranging between 50 and 300 nm, preferably between 100 and 250 nm, in an amount ranging between 0.001% and 10% of the total mass of the preparation; and
ii. at least one pharmaceutically acceptable excipient.

Typically, said preparation is in a form selected from the group consisting of gels, creams, lotions, sprays and ointments.

Typically, the excipient is selected from the group that comprises gelling agents, waxes, oils, surfactants, colorants, propellants, opacifiers, emollients, humectants, preservatives, antioxidants, emulsifiers, suspending agents, penetration enhancers and plasticizers.

### BRIEF DESCRIPTION OF THE ACCOMPANYING DRAWINGS

The disclosure will now be explained in relation to the non-limiting accompanying drawings, in which:

FIG. 1 illustrates the Field Emission Scanning Electron Microscopy (FESEM) images of barium zirconium oxide (BaZrO₃.BaCO₃) nanoparticles, where

1a represents the image at 20,000× magnification and

1b represents the image at 80,000× magnification

]FIG. 2 illustrates the X-ray diffractogram of BaZrO₃.BaCO₃ nanoparticles by X-ray Diffractometry (XRD).

FIG. 3 illustrates the relation between % X-ray absorbance and thickness (μm) of an X-ray retardant article.

### DETAILED DESCRIPTION

In accordance with one aspect of the present disclosure, there are provided nanoparticles of barium zirconium oxide (BaZrO₃.BaCO₃) having particle size ranging between 50 and 300 nm, preferably between 100 and 250 nm. The X-ray diffraction pattern of the nanoparticles have 20 values at 21.17, 30.06, 37.19, 43.16, 53.46, 62.62, 71.14 and 79.15.

In accordance with another aspect of the present disclosure, there is provided a process for synthesizing the BaZrO₃.BaCO₃ nanoparticles. The nanoparticles are synthesized by combustion method where, initially, a source of barium, a source of zirconium, a combustion agent, a thermal decomposition agent, a precipitation agent and a reducing agent are admixed under inert atmosphere. The source of barium is selected from a group that includes barium nitrate, barium chloride and barium acetate and is preferably barium nitrate. Similarly, the source of zirconium is selected from the group that includes zirconium oxychloride, zirconium chloride and zircolyl nitrate, preferably zirconium oxychloride. The source of barium and the source of zirconium are taken in the molar ratio of 2:1. The combustion agent that aids the overall combustion process, is selected from the group comprising ammonium nitrate, hydroxylammonium nitrate and hexanitrodiphenylamine, and is preferably ammonium nitrate. The thermal decomposition agent is selected from the group comprising glycine and N-methylglycine and is preferably glycine. The thermal decomposition agent is used to initiate and accelerate the combustion reaction. The precipitation agent, that is used for precipitating the nanoparticles, is selected from the group comprising urea, 1,3-dimethylurea and isopropylideneurea, and is preferably urea; whereas, the reducing agent is at least one selected from the group comprising starch, acrylamide and carboxymethylcellulose and is preferably starch. The quantities of the combustion agent, the thermal decomposition agent and the precipitation agent are in 1:1:5 proportion. The amount of reducing agent used in the reaction ranges between 10 and 30 mole %, preferably between 15 and 25 mole % with respect to the total moles of the source of barium. The process of admixing the reactants is carried out under inert atmosphere, preferably carbon dioxide (CO₂) atmosphere, which results in the formation of semi-transparent slurry. The slurry is further subjected to combustion by introducing it into a hot silica tube in a drop-wise fashion at a temperature ranging between 500° C. and 800° C., preferably between 645° C. and 655° C. The process of combustion is carried out for a time period ranging between 1.0 and 5.0 hours, preferably between 2.5 and 3.5 hours to yield the product in the form of porous and fluffy Ba₂ZrO₃.BaCO₃nanoparticles. The resultant nanoparticles, generally, have particle size that ranging between 50 and 300 nm, preferably between 100 and 250 nm.

In accordance with another aspect of the present disclosure there is provided a coating formulation chiefly comprising BaZrO₃.BaCO₃ nanoparticles, prepared by the combustion process. Along with the nanoparticles, the formulation further comprises a color guard and a vehicle. The BaZrO₃.BaCO₃ nanoparticles have particle size ranging between 50 and 300 nm, preferably between 100 and 250 nm. They are present in an amount ranging between 10% and 50%, preferably between 20% and 30% of the total mass of the vehicle. The color guard, along with carrying out its primary function of guarding the color pigment, also functions as a binder. Therefore, addition of the color guard thus enhances the property of the coating formulation to bind to various articles and objects it is contacted with. The color guard is selected from the group comprising light aliphatic solvent naphtha (64742-89-8), n-hexane(110-54-3), xylene (1330-207), acetone, ethyl benzene (100-41-4), silica amorphous, fumed, crystalline free (1129455-52-5), carbon black (133-86-4) [Loctite make], epoxy, nitrocellulose and ethyl cellulose and is included in an amount ranging between 5% and 25%, preferably between 8% and 12% of the total mass of the vehicle. The vehicle is selected from the group comprising ethyl methyl ketone, amyl acetate and acetone, preferably ethyl methyl ketone. The resultant coating formulation, chiefly due to the presence of BaZrO₃.BaCO₃nanoparticles, possesses X-ray retarding properties.

In accordance with yet another aspect of the present disclosure there is provided a process for the preparation of the coating formulation. As per the process, BaZrO₃.BaCO₃ nanoparticles are initially milled to yield a mass. The resultant mass is subsequently admixed with the afore-stated color guard and vehicle. The admixing is further followed by milling to yield the desired coating formulation. The process of milling is carried out for a time period ranging between 12 and 48 hours, preferably between 21 and 26 hours and is achieved by at least one method selected from the group that includes ball milling, is hammer milling, rod milling and tower milling. The coating formulation, predominantly due to its X-ray retardant properties, is used for coating various articles in order to render them X-ray retardant.

In accordance with yet another aspect of the present disclosure there is provided a process for the preparation of X-ray retardant articles. According to the process, initially, the article to be coated is contacted with the coating formulation possessing X-ray retardant properties. The article is selected from the group comprising aprons, gowns, scrubs, uniforms, gloves, caps, masks, curtains, sheets, fabrics, shoe covers, drapes, surgical pads, protective screens, thyroid collars, thyroid shields desks, drawers, rooms, walls, partitions, panels, tables, chairs, cabinets and ceiling panels. The article may be made of different materials selected from the group that includes cotton, epoxy resin, acrylic, lyocell, nylon, polyester, rubber, wood, glass, metal, metal alloys and paper. Different means may be adopted for the contacting process such as applying, spraying, dipping, incorporating, brushing and painting. Generally, the article is contacted with the formulation by the dip coating method. Next, the coated article is annealed by exposing it to hot air at a temperature ranging between 20° C. and 80° C., preferably between 50° C. and 60° C., in order to ensure particle adhesion to the surface. Annealing is carried out for a time period ranging between 0.5 and 5 minutes, preferably between 0.5 and 2 minutes, to finally yield the X-ray retardant article. Such an X-ray retardant article has applications in a variety of facilities such as an operation theatre, a radiology clinic, a dentists' clinic, a research laboratory and the petroleum industry.

In one embodiment of the present disclosure, the coating formulation may also be used as a paint where said paint, optionally in the presence of additives, may be applied on walls, partitions, floors and ceilings of facilities such as radiology clinics or radiology labs, which renders the facilities, X-ray retardant.

In accordance with still another aspect of the present disclosure, there is provided an X-ray retardant preparation that comprises BaZrO₃.BaCO₃ nanoparticles along with a pharmaceutically acceptable excipient. The nanoparticles have particle size ranging between 50 and 300 nm, preferably between 100 and 250 nm and are present in an amount ranging between 0.001% and 10% of the total mass of the preparation. The excipient is selected from the group comprising gelling agents, waxes, oils, surfactants, colorants, propellants, opacifiers, emollients, humectants, preservatives, antioxidants, emulsifiers, suspending agents, penetration enhancers and plasticizer. The X-ray retardant preparation may be formulated in different forms such as gels, creams, lotions, sprays and ointments.

The present disclosure is further described in light of the following examples which are set forth for illustration purpose only and not to be construed for limiting the scope of the disclosure.

### EXAMPLE 1

### Synthesis of Barium Zirconium Oxide (BaZrO₃.BaCO₃) Nanoparticles

Two moles of the barium nitrate (99%) and one mole of zirconium oxychloride were mixed with ammonium nitrate, glycine, urea and starch under carbon dioxide atmosphere in a mortar to form semi-transparent slurry. The proportion of ammonium nitrate, glycine, and urea was 1:1:5. 0.2 g (0.01M) of barium nitrate of starch was added. Further, the slurry was slowly added in a drop-wise fashion into a hot silica tube, maintained at 650° C. BaZrO₃.BaCO₃ nanoparticles were obtained in the silica tube in the form of a porous and fluffy material.

### EXAMPLE 2

### Preparation of the Coating Formulation

25 g of BaZrO₃.BaCO₃ nanoparticles were ball milled up to 24 hours. The ball milled powder was mixed with 10 g of an admixture of light aliphatic solvent naphtha (64742-89-8), n-hexane(110-54-3), xylene (1330-207), acetone, ethyl benzene (100-41-4), silica amorphous, fumed, crystalline free (1129455-52-5) carbon black (133-86-4) [Loctite make] Color Guard R, that was used as a color guard and 100 ml of ethyl methyl ketone that was used as a vehicle. The mixture was further ball milled for up to 24 hours to result in the coating formulation.

### EXAMPLE 3

### Fabrication of Coated Aprons

The coating formulation obtained from Example 2 was coated on a cotton cloth by dip coating method. Said coating was annealed at 60° C., for 1.5 minutes by employing hot air to provide two aprons, one small sized (2.5×3.5 cm) and the other large sized (30×40 cm) aprons were prepared.

### EXAMPLE 4

### Characterization of BaZrO₃.BaCO₃ Nanoparticles

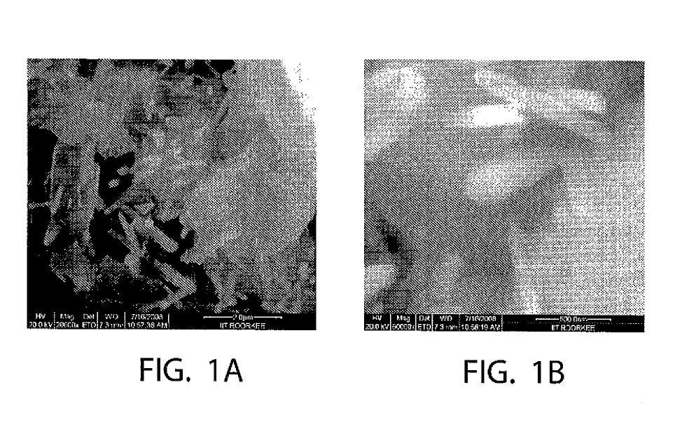

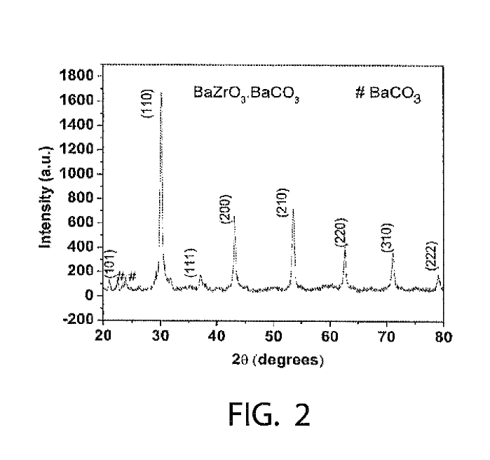

A] FESEM analysis: Morphological study of the BaZrO₃.BaCO₃ nanoparticles was performed by FESEM and recorded on JEOL instrument (IIT Roorkee). The images obtained from FESEM demonstrated rod-like morphology and the particle size (diameter of rod) was found to be ranging between 100-250 nm (FIG. 1).

B] XRD analysis: X-ray diffraction (XRD) analysis of the BaZrO₃.BaCO₃ nanoparticles was carried out to reveal sharp and distinct peaks, indicating crystalline nature of the compound (FIG. 2). In addition to the presence of the peaks of BaZrO₃, a couple of peaks of BaCo₃ were also observed. Indexing of the compound was carried out with the help of X'pert high score plus software. Further, the compound also showed a cubic structure where the unit cell parameters were found to be a=b=c=4.1930 A°

The X-ray diffraction pattern of the BaZrO₃.BaCO₃ nanoparticles showed 20 values at 21.17, 30.06, 37.19, 43.16, 53.46, 62.62, 71.14 and 79.15.

### EXAMPLE 5

### Qualitative Evaluation of the Coated Article

The small sized apron prepared from Example 3 was independently exposed to a dental (up to 40 Kv) as well as an orthopedic (60 Kv) X-ray source for qualitative evaluation purposes.

No change was observed in the apron post-exposure to the X-rays.

### EXAMPLE 6

### Quantitative Evaluation of the Coated Article

Three samples were prepared from the large sized apron provided by Example 3. The first sample bore a single coating of the coating formulation prepared from Example 2, the second one bore a two-fold coating whereas the third one bore of four-fold coating. All three samples, along with a control, were exposed to an X-ray source (60 Kv and 80 Kv) and testing machine provided by Shonal Medical Equipments Pvt. Ltd. The X-ray source/tube was obtained from Bharat Electronics (BEL) Pune; whereas the Leakage Radiation Meter, that formed a part of the testing machine, was obtained from PTW, Germany. Quantitative X-ray absorbance as well as X-ray penetration with respect to the thickness of the coated cloth was tested. The results obtained are as follows:

TABLE 1
X-ray absorption at 60 Kv potential and 48.6 smAs voltage.

| | | Thickness | | | |
|---|---|---|---|---|---|
| | | of | | | |
| | | the coated | X-ray | X-ray | X-ray |
| Sr. | | material | transmitted | absorbance | absorbance |
| No. | Parameter | (μm) | (μGray) | (μGray) | (%) |
| 1 | Direct | — | 1022 | — | — |
| | exposed | | | | |
| 2 | Single | 550 | 491.1 | 530.9 | 51.94 |
| 3 | Two fold | 1100 | 252.5 | 769.5 | 75.29 |
| 4 | Four fold | 2200 | 101.4 | 920.6 | 90.78 |

TABLE 2
X-ray absorption at 80 Kv potential and 64.4 mAs voltage.

| | | Thickness | | | |
|---|---|---|---|---|---|
| | | of | | | |
| | | the coated | X-ray | X-ray | X-ray |
| Sr. | | material | transmitted | absorbance | absorbance |
| No. | Parameter | (μm) | (μGray) | (μGray) | (%) |
| 1 | Direct | — | 1903 | — | — |
| | exposed | | | | |
| 2 | Single | 550 | 1009 | 894 | 46.97 |
| 3 | Two fold | 1100 | 612.4 | 1290.6 | 67.81 |
| 4 | Four fold | 2200 | 295.4 | 1607.6 | 84.47 |

The value of X-ray absorbance for the samples was calculated by subtracting the value of X-ray transmittance of the samples from the value of X-ray transmittance of the directly exposed apron.

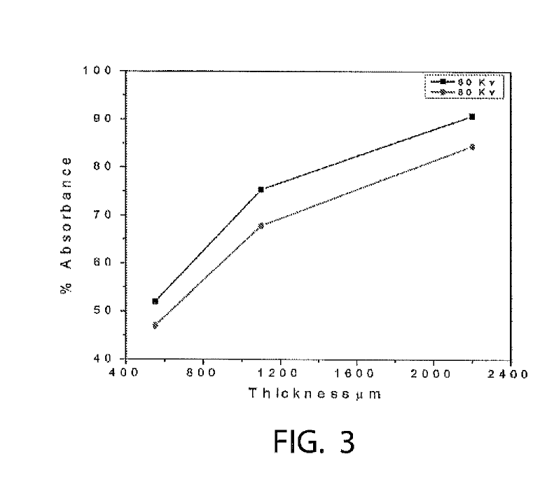

It was observed that X-ray absorbance increased with increase in thickness (FIG. 3). Thus, at 60 Kv, 90.78% and at 80 Kv, 84.47% of the X-rays were absorbed.

Technical Advantages

The present disclosure, related to barium zirconium oxide (BaZrO₃.BaCO₃) nanoparticles, has the following technical advantages:
(1) provides a cost-efficient, rapid and high yielding process for the preparation of BaZrO₃.BaCO₃ nanoparticles,
(2) provides a less toxic coating formulation possessing X-ray attenuation properties,
(3) provides a simple and economical process for the preparation of a coating formulation, and
(4) provides highly effective X-ray retardant articles and preparations.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

The numerical values given for various physical parameters, dimensions and quantities are only approximate values and it is envisaged that the values higher than the numerical value assigned to the physical parameters, dimensions and quantities fall within the scope of the invention and the claims unless there is a statement in the specification to the contrary.

While certain embodiments of the inventions have been described, these embodiments have been presented by way of example only, and are not intended to limit the scope of the inventions. Variations or modifications in the process or compound or formulation or combination of this invention, within the scope of the invention, may occur to those skilled in the art upon reviewing the disclosure herein. Such variations or modifications are well within the spirit of this invention. The accompanying claims and their equivalents are intended to cover such forms or modifications as would fall within the scope and spirit of the invention.

1. Nanoparticles of barium zirconium oxide (BaZrO3.BaCO3); said nanoparticles characterized by particle size ranging between 50 and 300 nm, preferably between 100 and 250 nm and the X-ray diffraction pattern having 20 values at 21.17, 30.06, 37.19, 43.16, 53.46, 62.62, 71.14 and 79.15.

2. A process for synthesizing barium zirconium oxide (BaZrO3.BaCO3) nanoparticles as claimed in claim 1; said process comprising the following steps:
i. admixing at least one source of barium selected from the group consisting of barium nitrate, barium chloride and barium acetate, at least one source of zirconium selected from the group consisting of zirconium oxychloride, zirconium chloride and zircolyl nitrate, at least one combustion agent selected from the group consisting of ammonium nitrate, hydroxylammonium nitrate and hexanitrodiphenylamine, at least one thermal decomposition agent selected from the group consisting of glycine and N-methylglycine, at least one precipitation agent and at least one reducing agent under inert atmosphere to obtain a slurry; and
ii. subjecting said slurry to combustion at a temperature ranging between 500° C. and 800° C., preferably between 645° C. and 655° C., for a time period ranging between 1.0 and 5.0 hours, preferably between 2.5 and 3.5 hours to obtain BaZrO3.BaCO3 nanoparticles.

3. The process as claimed in claim 2, wherein the mole ratio of the source of barium to the source of zirconium is 2:1.

4. The process as claimed in claim 2, wherein the precipitation agent is at least one selected from the group consisting of urea, 1,3-dimethylurea and isopropylideneurea, preferably urea.

5. The process as claimed in claim 2, wherein the reducing agent is at least one selected from the group consisting of starch, acrylamide and carboxymethylcellulose, preferably starch, in an amount ranging between 10 and 30 mole %, preferably between 15 and 25 mole % with respect to the total moles of the source of barium.

6. The process as claimed in claim 2, wherein the proportion of the combustion agent, the thermal decomposition agent and the precipitation agent is 1:1:5.

7. The process as claimed in claim 2, wherein the inert atmosphere is a carbon dioxide (CO2) atmosphere.

8. The process as claimed in claim 2, wherein the method-step of combustion involves addition of said slurry into a hot silica tube in a drop-wise fashion.

9. A coating formulation comprising barium zirconium oxide (BaZrO3.BaCO3) nanoparticles having particle size ranging between 50 and 300 nm, preferably between 100 and 250 nm, in an amount ranging between 10% and 50%, preferably between 20% and 30% of the total mass of a vehicle, at least one color guard and at least one vehicle selected from the group comprising ethyl methyl ketone, amyl acetate and acetone.

10. The coating formulation as claimed in claim 9, wherein the color guard is at least one selected from the group consisting of light aliphatic solvent naphtha (64742-89-8), n-hexane(110-54-3), xylene (1330-207), acetone, ethyl benzene (100-41-4), silica amorphous, fumed, crystalline free (1129455-52-5) carbon black (133-86-4) [Loctite make], epoxy, nitrocellulose and ethyl cellulose in an amount ranging between 5% and 25%, preferably between 8% and 12% of the total mass of the vehicle.

11. A process for the preparation of a coating formulation as claimed in claim 9 said process comprising the following steps:
i) milling said BaZrO3.BaCO3 nanoparticles in an amount ranging between 10% and 50%, preferably between 20% and 30% of the total mass of the vehicle for a time period ranging between 12 and 48 hours, preferably between 21 and 26 hours to obtain a mass; and
ii) admixing said mass and the color guard in an amount ranging between 5% and 25%, preferably between 8% and 12% of the total mass of the vehicle, in the vehicle, followed by milling to obtain said coating formulation.

12. A process for the preparation of an X-ray retardant article; said process comprising the following steps:
i. contacting said article with a coating formulation as claimed in claim 9 by at least one method selected from the group comprising applying, spraying, dipping, incorporating, brushing and painting, to obtain a coated article; and
ii. annealing the coated article by hot air at a temperature ranging between 20° C. and 80° C., preferably between 50° C. and 60° C. for a time period ranging between 0.5 and 5 minutes, preferably between 0.5 and 2 minutes obtain an X-ray retardant article.

13. The process as claimed in claim 12, wherein the article is at least one selected from the group comprising aprons, gowns, scrubs, uniforms, gloves, caps, masks, curtains, sheets, fabrics, shoe covers, drapes, surgical pads, protective screens, thyroid collars, thyroid shields, desks, drawers, rooms, walls, partitions, panels, tables, chairs, cabinets and ceiling panels.

14. The process as claimed in claim 12, wherein the color guard is at least one selected from the group comprising light aliphatic solvent naphtha (64742-89-8), n-hexane(110-54-3), xylene (1330-207), acetone, ethyl benzene (100-41-4), silica amorphous, fumed, crystalline free (1129455-52-5) carbon black (133-86-4) [Loctite make], epoxy, nitrocellulose and ethyl cellulose, in an amount ranging between 5% and 25%, preferably between 8% and 12% of the total mass of the vehicle.

15. The process as claimed in claim 12, wherein the vehicle is at least one selected from the group comprising ethyl methyl ketone, amyl acetate and acetone, preferably ethyl methyl ketone.

16. An X-ray retardant article, prepared by the process as claimed in claim 12, wherein said article is at least one article selected from the group comprising aprons, gowns, scrubs, uniforms, gloves, caps, masks, curtains, sheets, fabrics, shoe covers, drapes, surgical pads, protective screens, thyroid collars, thyroid shields, desks, drawers, rooms, walls, partitions, panels, tables, chairs, cabinets and ceiling panels.

17. The X-ray retardant article as claimed in claim 12, wherein said article is made up of at least one material selected from the group consisting of cotton, epoxy resin, acrylic, lyocell, nylon, polyester, rubber, wood, glass, metal, metal alloys and paper.

18. An X-ray retardant preparation comprising:
i. barium zirconium oxide (BaZrO3.BaCO3) nanoparticles having particle size ranging between 50 and 300 nm, preferably between 100 and 250 nm, in an amount ranging between 0.001% and 10% of the total mass of the preparation; and
ii. at least one pharmaceutically acceptable excipient.

19. The preparation as claimed in claim 18, wherein said preparation is in a form selected from the group consisting of gels, creams, lotions, sprays and ointments.

20. The preparation as claimed in claim 18, wherein the excipient is selected from the group that comprises gelling agents, waxes, oils, surfactants, colorants, propellants, opacifiers, emollients, humectants, preservatives, antioxidants, emulsifiers, suspending agents, penetration enhancers and plasticizers.
